(19) United States
(12) Patent Application Publication
(10) Pub. No.: US 2008/0319549 A1
(43) Pub. Date: Dec. 25, 2008

(54) EXPANDABLE SUPPORT DEVICE AND METHOD OF USE

(75) Inventors: E. Skott GREENHALGH, Lower Gwynedd, PA (US); John-Paul ROMANO, Chalfont, PA (US)

Correspondence Address: LEVINE BAGADE HAN LLP, 2483 EAST BAYSHORE ROAD, SUITE 100, PALO ALTO, CA, 94303, US

(73) Assignee: Stout Medical Group, L.P., Perkasie, PA (US)

(21) Appl. No.: 12/139,378

(22) Filed: Jun. 13, 2008

Related U.S. Application Data
(63) Continuation of application No. PCT/US2006/049607, filed on Dec. 28, 2006.
(60) Provisional application No. 60/754,492, filed on Dec. 28, 2005.

(51) Int. Cl. A61F 2/44; A61M 29/00

(52) U.S. Cl. 623/17.16; 623/17.11; 606/191

(57) ABSTRACT

A device for separating a first bone from a second bone is disclosed. The device can be an expandable orthopedic jack. The device can be used to treat spinal stenosis. The device can be deployed between adjacent spinous processes and then increased in height to reduce pressure on nearby nerves. Methods for using the device are also disclosed.

### CROSS-REFERENCE TO RELATED APPLICATION

This application is a continuation of PCT International Application No. PCT/US2006/049607, filed Dec. 28, 2006 which claims the benefit of U.S. Provisional Application No. 60/754,4492, filed Dec. 28, 2005, which are incorporated herein by reference in their entireties.

### BACKGROUND OF THE INVENTION

This invention relates to devices for providing support for biological tissue, for example to repair spinal stenosis and/or spinal compression fractures, and methods of using the same.

Spinal stenosis is often caused by a shift in the vertebral bodies, which in turn change the static and dynamic nature of the spine. As the spine column shifts, load distributions change, tendons in the spine often shrink, and muscles reorganize and compensate. This can result in bone bumping into other bones. This can result in hypertrophy of the facet joints, or degenerative disc disease, which in turn can force the tissue surrounding the spinal cord and/or dorsal and ventral roots to compress and irritate the respective nerves. This irritation and compression can cause pain.

Over time this “downward spiral”, cascading process often gets worse. People with spinal stenosis may start to favor their spine, hunching over. This hunching can cause yet more load shifting, and more long term tissue damage and pain.

Existing mechanical treatment include a laminectomy, which removes the adjacent lamina and often a portion of the facet joints. Another procedure performed to treat spinal stenosis is a facetectomy, removing tissue from the facet joints, for example complete removal of the facet or partial removal using a rongeur. However, healthy tissue damage and destruction is required by either of these methods, whether used alone or in combination. Also, non-target tissue can be damaged, including spinal nerve tissue. Further this procedure is typically performed in an open surgery, requiring more damage and longer healing time.

Another treatment includes an attempt to mechanically restore adjacent vertebrae to an angle with respect to each other that will prevent the vertebrae from pinching the affected nerves. FIGS. 1 through 3 illustrate this concept. FIG. 1 illustrates that a first vertebra 102 can have a first vertebral plane 104. A second vertebra 106 can have a second vertebral plane 108. The first vertebra 102 can have a first vertebral goal plane 110. The first vertebral goal plane 110 is the plane at which the first vertebra 102 will not, or will minimally, press, pinch, or otherwise pathologically interfere with the surrounding nerves (e.g., spinal cord 112 or dorsal or ventral roots 114), such as shown at a compressed nerve area 116. The difference between the first vertebral plan 104 and the first vertebral goal plane 110 can be a vertebral angle 118. The first vertebral goal plane 110 and the second vertebral plane 108 can be substantially parallel.

The device 200 can be positioned near the treatment site, as shown in FIG. 1. The device may have a cam, or prop 202. The device can have straps or braces 204 to secure to the adjacent vertebra. FIG. 2 illustrates that the device 200 having a cam 202 can be inserted between the first and second vertebrae's' processes. FIG. 3 illustrates that the cam 204 can be turned to expand, as shown by arrows, pushing the dorsal ends of the vertebrae 102 and 106 apart. This rotates the first vertebra 102 so the first vertebral plane 102 becomes coplanar with the first vertebral goal plane 110. The affected nerve 116 will therefore be no longer compressed, or be less compressed.

One method of accomplishing this treatment includes the deployment of a static mechanical prop between vertebrae. The prop is used to wedge into place between adjacent vertebrae and push the adjacent vertebrae back to a naturally beneficial relative angle, often relieving the pressure on the affected nerve. The prop is commonly attached to the adjacent vertebrae using straps. However, the prop is not adjustable in height and the straps must be surgically attached around the adjacent vertebra.

Yet another existing prop has fixed lateral braces and an adjustable cam that separates the vertebrae. The fixed braces are significantly larger than the prop and require an open procedure to deploy, requiring significant additional tissue destruction and damage to deploy than the cam alone. Further, the cam has a relatively small range of expansion and produces an unnatural, significantly rigid connection between the adjacent vertebrae, much like the static prop.

A less invasive treatment option to regain support height between affected vertebrae is desired. A device that can produce a more natural mechanical resolution of the altered angle between adjacent vertebrae is also desired. Further, a device is desired that can be adjusted in vivo to the desired height between adjacent vertebrae.

### SUMMARY OF THE INVENTION

A method is disclosed that can include implanting an expandable support device between adjacent bones, such as vertebrae. This less invasive treatment method can increase height in the spine and provide mechanical support in the spine. This method and the associated device can reduce trauma to the soft tissue and reduce the disruption to the ligaments in the spine, increasing spinal stability. The expandable support device can be used as a spinal lift device. The expandable support device can also be used as an expandable space creator, for example between two or more bones, such as vertebra.

A method for treating spinal stenosis is disclosed. The method can include positioning an expandable support device between a first vertebra and a second vertebra, where the first vertebra is adjacent to the second vertebra. The method can also include compressing the expandable support device.

Compressing can include applying a compressive force in a first direction. Compressing can also include expanding the expandable support device in a second direction. The second direction can be substantially perpendicular to the first direction.

Compressing can include applying a compressive force along an axis that is substantially perpendicular to a line from an anatomical landmark on the first vertebra to the anatomical landmark on the second vertebra. Compressing can include expanding the height of the expandable support device. The height can be measured along an axis that is substantially parallel with a line from an anatomical landmark on the first vertebra to the anatomical landmark on the second vertebra.

The method can also include sensing the compressed expandable support device, then further compressing the compressed expandable support device. Sensing can include visualizing, such as by MRI, CT scan, radiocontrast visualization, direct visualization, fiber optic visualization, or combinations thereof. The method can also include further expanding the expandable support device after initially expanding and visualizing the expandable support device.

An expandable support device for treating spinal stenosis by applying substantially oppositely directed forces on a first bone and a second bone is also disclosed. The device can have an expandable frame. The expandable frame can have a first elongated element, a second elongated element, and a first connector, such as an end plate. The first elongated element can have a first elongated element first end and a first elongated element second end. The second elongated element can have a second elongated element first end and a second elongated element second end. The first connector can connect the first elongated element to the second elongated element. The expandable frame can be configured to expand in a first direction when the expandable frame is compressed in a second direction.

The first elongated element and the second elongated element can interdigitate.

The device can have a second connector connecting the first elongated element to the second elongated element. The first connector can be connected to the first elongated element at the first elongated element first end. The second connector can be connected to the first elongated element at the first elongated element second end. The connection between the first elongated element and the first connector can include the first connector being integral with the first elongated element.

The first connector can be configured to attach to a compression tool. The second connector can be configured to attach to the compression tool.

The expandable frame can be configured to bend about an axis substantially parallel with the first direction. The expandable frame can be configured to bend about an axis substantially perpendicular to the first direction and the second direction.

The first elongated element can have a seat configured to attach to the first bone, and wherein the seat is configured in a different shape than the adjacent portion of the first elongated element.

### BRIEF DESCRIPTION OF THE DRAWINGS

FIGS. 1 through 3 illustrate a generic method for treating spinal stenosis by mechanically rotating and supporting a vertebra. The variation of the device is shown schematically.

FIGS. 4a and 4b illustrate variations of the expandable support device in a contracted configuration.

FIG. 5 illustrates the variation of the expandable support device of FIG. 4a or 4b in an expanded configuration, not to scale.

FIG. 6a is a side view of a variation of the expandable support device in a contracted configuration.

FIG. 6b is a perspective view of the expandable support device of FIG. 6a.

FIG. 7a is a side view of the expandable support device of FIG. 6a in an expanded configuration.

FIG. 7b is a perspective view of the expandable support device of FIG. 6a in an expanded configuration.

FIG. 8 illustrates a variation of the expandable support device in a contracted configuration.

FIGS. 9 and 10a are perspective views of variations of the expandable support device.

FIG. 10b is a side view of a variation of the expandable support device of FIG. 10a.

FIGS. 11a and 11b illustrate a variation of a method for using a variation of the expandable support device.

FIGS. 12a and 12b illustrate a variation of a method for using a variation of the expandable support device.

FIGS. 13a and 13b illustrate a variation of a method for using a variation of the expandable support device.

FIG. 14 illustrates a variation of the expandable support device deployed in a spine.

FIG. 15 is a close-up view of a portion of a variation of the expandable support device deployed in a spine.

FIG. 16a is a top view of a variation of the expandable support device during deployment in a spine.

FIG. 16b is a front view of FIG. 16a with different anatomical features shown.

FIG. 17a is a top view of the expandable support device of FIG. 16a further along during deployment in a spine.

FIG. 17b is a front view of FIG. 17a with different anatomical features shown.

FIG. 18 illustrates variations of methods for deploying the expandable support device.

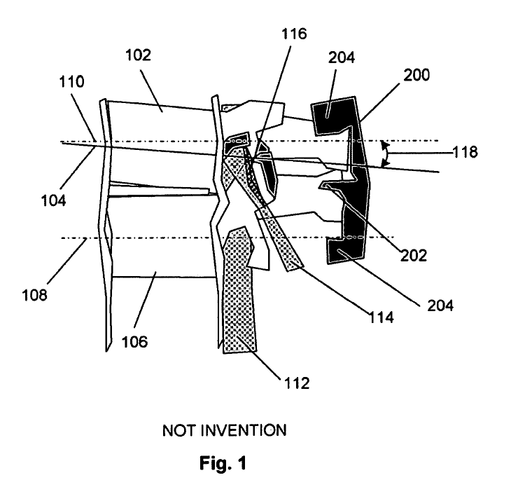

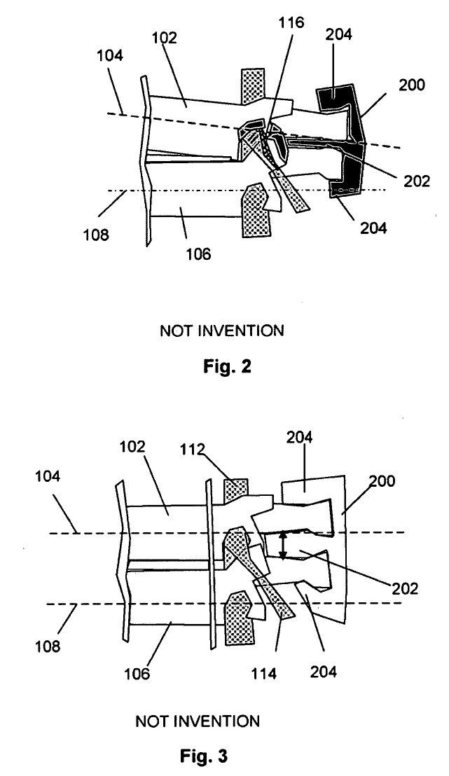

### DETAILED DESCRIPTION

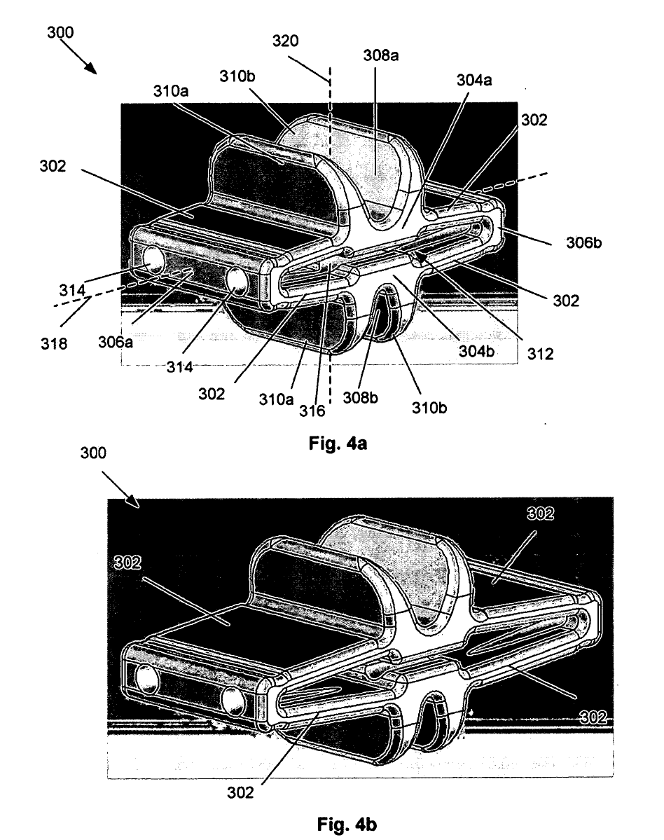

FIGS. 4a and 4b illustrates that the expandable support device 300 can have an expandable and compressible frame. FIGS. 4a and 4b illustrate the expandable support device in a radially contracted (i.e., flattened, height contracted) configuration.

The expandable support device 300 can have two, three, four or more struts The struts 302 can be rotationally connected to (i.e., attached to or intregrated with) some or all of the other struts 302. The expandable support device 300 can have a top plate 304 and/or a bottom plate 306. The plates 304 can be rotationally connected to one, some or all of the struts 302. The expandable support device 300 can have a first end plate 306a and/or a second end plate 306b. The struts 302 and/or plates 304 and/or 306 can rotationally connect to any or all of each other.

The struts 302 and/or plates 304 can have a first vertebral seat 308a and/or a second vertebral seat 308b. The first and second vertebral seats 308a and 308b can be configured to attach to the first and second vertebrae 102 and 106, respectively. The vertebral seats 308 can be configured to minimize or completely prevent lateral movement of the vertebrae 102 and 106. For example, the seats 308 can each have a seat first side 310a and/or a seat second side 310b. The seat first side 310a can form a right or acute angle with the seat second side 310b. The vertebral seats 308 can have a “V” configuration.

The struts 302 and/or plates 304 and/or 306 can form one or more channels or holes 312. One or both of the end plates 306 can have one, two or more tool interfaces, such as tool interface ports 314. The tool interface ports 314 can be configured to removably attach to a deployment tool. The struts 302 and/or plates 304 and/or 306 can have grooves 316 to receive a deployment tool and/or locking element (e.g., to resist expansion and/or contraction of the expandable support device 300).

The expandable support device 300 can have a compression or longitudinal axis 318. The expandable support device can have an expansion axis 320. The compression axis 318 can be perpendicular to the expansion axis 320. The compression axis 318 can be parallel with the deployment tool interface ports 314.

FIG. 4b illustrates that the dimensions of the expandable support device 300 and the elements thereof can vary from those of FIG. 4a, even with a similar configuration. The expandable support device 300 can be configured to fit a particular patient anatomy. For example, a physician could select from a number of variously sized expandable support devices to best fit the patient.

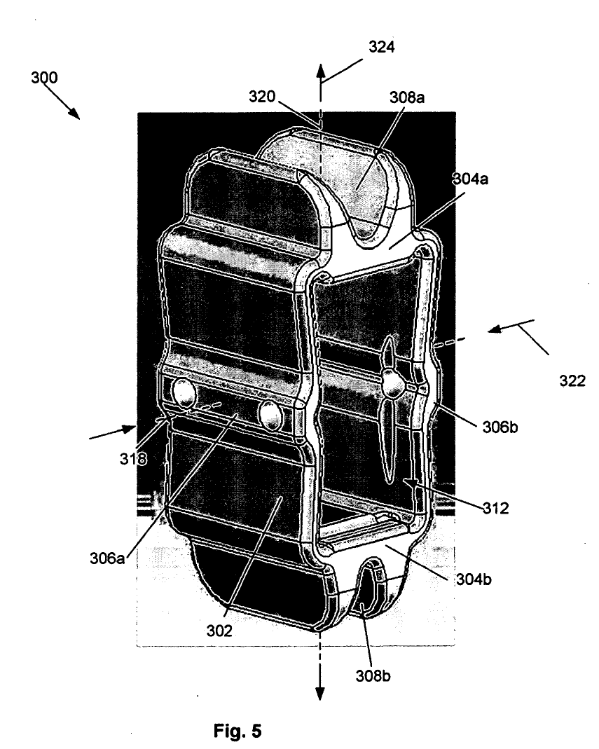

FIG. 5 illustrates that the expandable support device 300 can be in a radially expanded (i.e., radially expanded, heightened) configuration. A compression force, as shown by arrows 322, can be applied along the compression axis 318. The compression force can cause rotation of the struts 302 with respect to each other, and the plates 304 and 306. The compression force can cause expansion, as shown by arrows 324, of the expandable support device 300 along the expansion axis 320. The expansion can result in the first and second vertebra seats 308a and 308b translating away from each other.

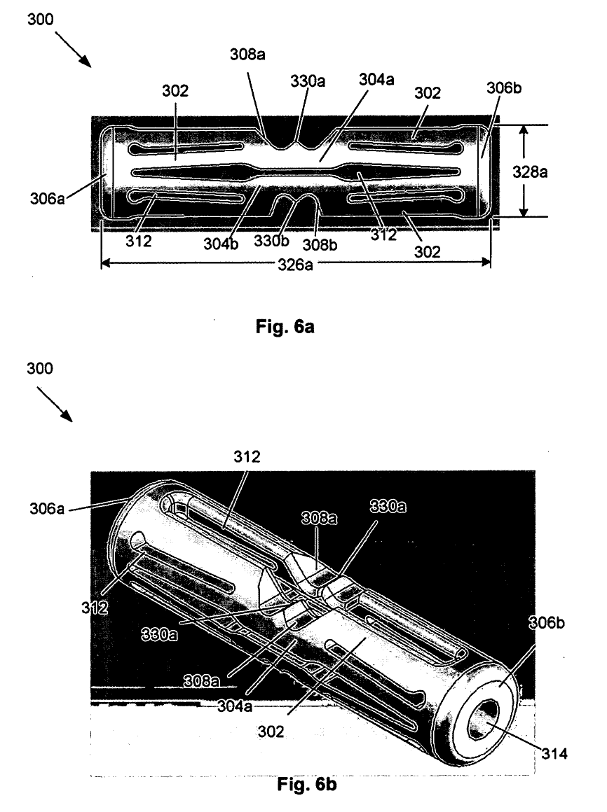

FIGS. 6a and 6b illustrate that the expandable support device 300 can have an expandable support device contracted length 326a and an expandable support device contracted height 328a. The expandable support device contracted length 326a can be from about 16 mm (0.63 in.) to about 66 mm (2.6 in.), for example about 33 mm (1.3 in.). The expandable support device contracted height 328a can be from about 4 mm (0.2 in.) to about 16 mm (0.63 in.), for example about 8 mm (0.3 in.).

The vertebral seats 308 can have seat anchors 330. The seat anchors 330 can attach to the bone in the vertebral seat 308 during use. The seat anchor 330 can restrict lateral and/or posterior/anterior movement of the bone. The seat anchors 330 can have points, ridges, hooks, barbs, brads, or combinations thereof. The vertebral seats 308 can have a “W” configuration.

The expandable support device 300 can have a generally cylindrical configuration, for example in the contracted configuration. The end plates 306 can be substantially circular or oval. The end plates 306 can each have a single deployment tool port 314. The deployment tool ports 314 can be substantially centered on the end plates 306.

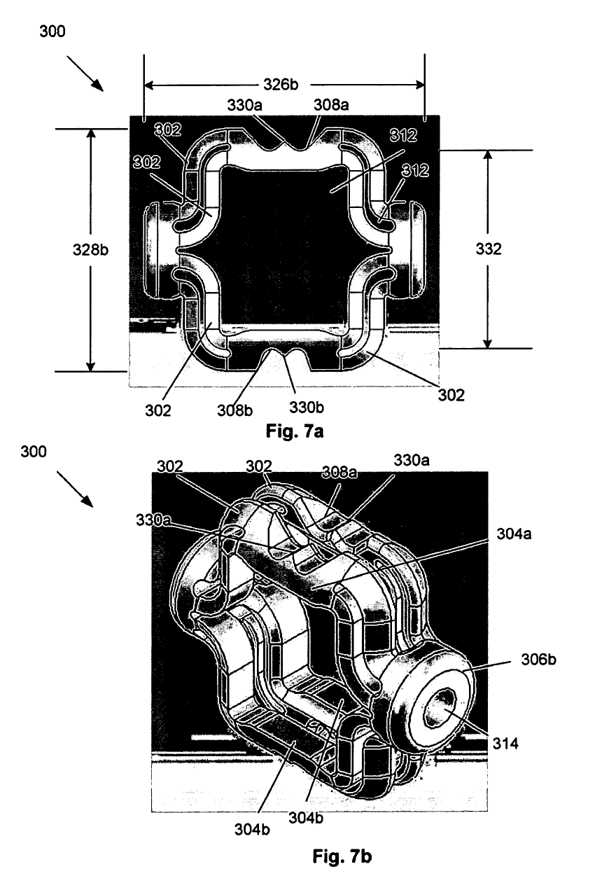

The expandable support device 300 can have two or more rows of completely or substantially parallel struts 302 and/or plates 304 in the longitudinal direction. The first and/or second vertebral seats 308a and/or 308b can each be on a single strut 302 or plate 304, or can be split onto two or more struts 302 and/or plates 304, as shown in FIGS. 6b and 7b.

FIGS. 7a and 7b illustrate that the expandable support device 300 can have an expandable support device expanded length 326b and an expandable support device expanded height 328b. The expandable support device expanded length 326b can be from about 11 mm (0.43 in.) to about 46 mm (1.8 in.), for example about 23 mm (0.91 in.). The expandable support device expanded height 328b can be from about 10 mm (0.39 in.) to about 40 mm (1.6 in.), for example about 20 mm (0.79 in.).

The expandable support device can have an expanded seat height 332. The expanded seat height 332 can be the distance between the first vertebral seat 308a and the second vertebral seat 308b when the expandable support device 300 is in an expanded configuration. The expanded seat height 332 can be from about 8 mm (0.3 in.) to about 33 mm (1.3 in.), for example about 16.5 mm (0.650 in.).

In the expanded configuration, the expandable support device 300 can form acute, and/or obtuse, and/or substantially right angles between the struts 302, and plates 304 and 306. For example, the side view (longitudinal cross-section) can be substantially rectangular and/or square, as shown in FIG. 7a.

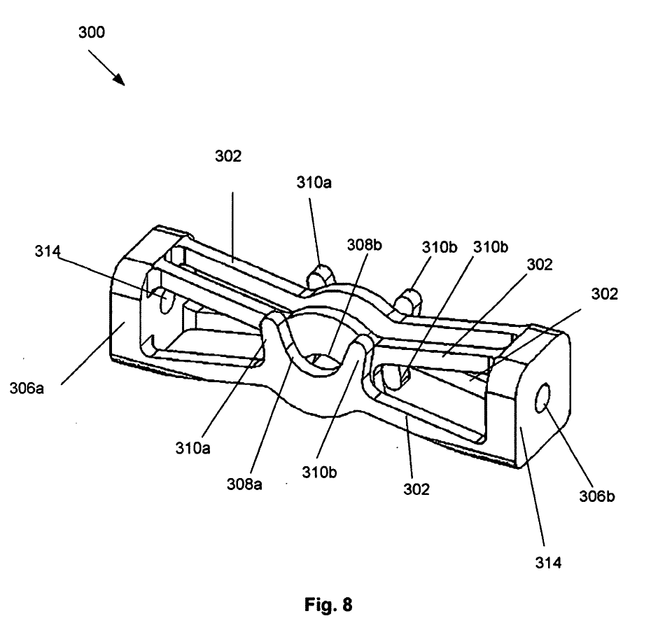

FIG. 8 illustrates that the expandable support device can have interdigitating struts 302. The vertebral seats 308 can have a “C” or “U” configuration. The end plates 306 can have substantially square configurations.

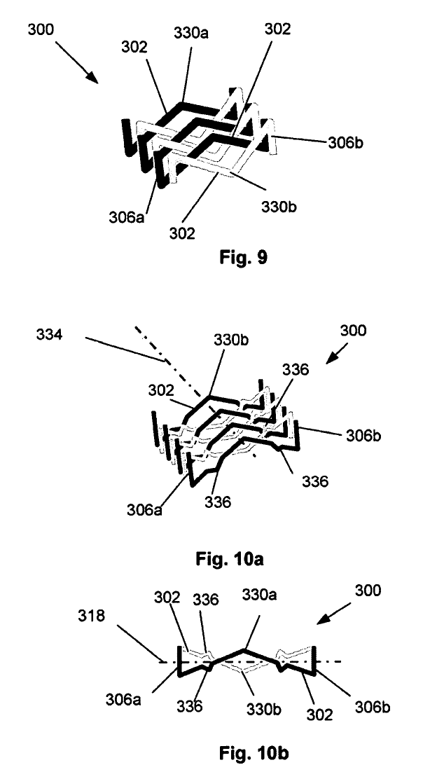

FIG. 9 illustrates that the expandable support device can have no vertebral seats 308. Adjacent struts 302 can join to form a vertebral anchor 330. Between the plates 306a and 306b, the expandable support device 330 can be entirely straight struts 302. The end plates 306a can be individual and separated for each strut 302, and/or flexibly joined together.

FIG. 9 illustrates that the expandable support device can have a transverse axis 334. The transverse axis 334 can be perpendicular to the longitudinal axis 318 and/or expansion axis 320.

FIGS. 9 and 10 illustrate that the struts 302 (as shown), or plates 304 can have length adjusters 336. The length adjusters 336 can contract and expand, for example to fit the length of the expandable support device 300 to the length of the target site, also for example, to ease introduction of the expandable support device 300 through soft and hard tissue when being inserted to the target site. The length expanders 336 can be hinges, springs, or combinations thereof. The length expanders 336 can be configured to rotate, and/or expand, and/or contract. The length expanders 336 can be attached to, and/or integral with the adjacent struts 302 and/or plates 304.

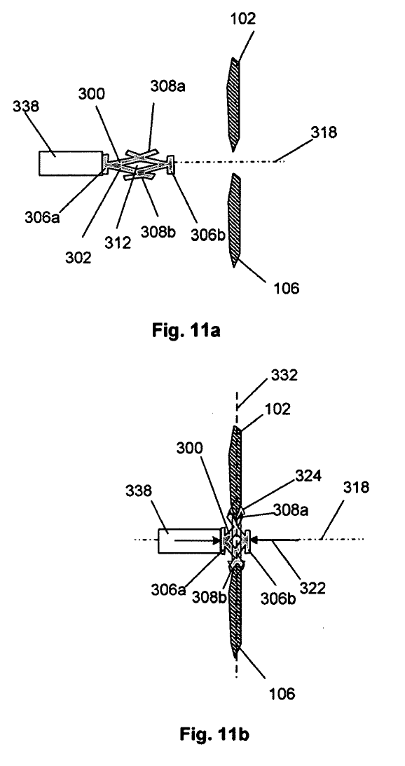

FIG. 11a illustrates that the expandable support device 300 can be inserted to the target site attached to a deployment tool 338. The deployment tool 338 can be part of a delivery system (not shown) that can include a catheter, trocar, drill, balloon, or a combination thereof. The deployment tool 338 can follow a guide wire into position between the tilted spinous process (e.g., of the stenotic vertebra 102 and 106) and deployed.

The deployment tool 338 can be attached to the expandable support device 300 via the deployment tool interface ports 314. The deployment tool 338 can extend through and/or around the length of the expandable support device 300. The deployment tool 338 can attach to the distal and/or proximal ends of the expandable support device 300, for example to deploy a compressive or tensile force to the expandable support device 300 along the compression or longitudinal axis 318.

The expandable support device 300 can be inserted into the target site, for example along the longitudinal axis 318. The expandable support device 300 can be inserted into the target site in an orietantion perpendicular to the longitudinal axis 318, for example, the expandable support device 300 shown in FIGS. 4a, 4b and 5.

FIG. 11b illustrates that when the expansion axis is aligned with the vertebrae 102 and 106, for example at the spinous processes, and/or when the vertebral seats 308 are aligned with the closest points of the vertebrae 102 and 106 (e.g., the closest points of the spinous processes), then the deployment tool 338 can compress, as shown by arrows 322, the expandable support device 300 along the compressive or longitudinal axis 318. The expandable support device 300 can then expand, as shown by arrows 324, in height along the expansion axis 332.

As the expandable support device 300 expands in height, the expandable support device contacts the first and second vertebrae 102 and 106. The first and second vertebrae 102 and 106 can attach to the expandable support device 300, for example, at the first and second vertebral seats 308a and 308b, respectively.

As the expandable support device 300 is continued to be compressed, and therefore continued to be expanded in height, the first vertebrae 102 can be forced away from the second vertebra 106, for example, at the spinous processes, thereby rotating and/or translating the first vertebra 102 with respect to the second vertebra The rotation and/or translation of the first vertebra 102 with respect to the second vertebra 106 can decompress the affected nerve.

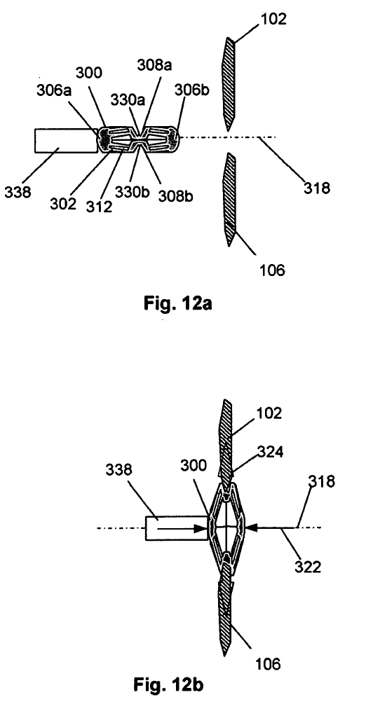

FIGS. 12a and 12b illustrate deployment and expansion of the expandable support device 300 similar to the expandable support device 300 shown in FIGS. 6a, 6b, 7a and 7b. The vertebral anchors 330 can attach to, and press in to the vertebrae 102 and 106 during expansion of the expandable support device 300.

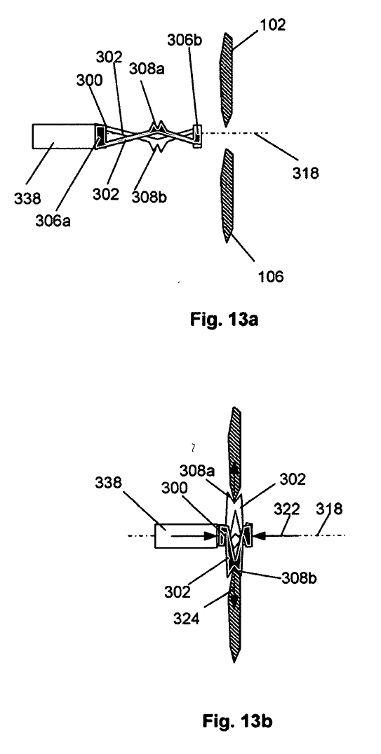

FIGS. 13a and 13b illustrate deployment and expansion of the expandable support device 300 similar to the expandable support device 300 shown in FIG. 8. When deployed into an expanded configuration, the interdigitating struts 302 can rotate toward the same or opposite directions during deployment as the initial starting position of the strut 302 in the contracted configuration. For example, even though a first strut can be on a first side (e.g., top) and a second strut can be on a second side (e.g., bottom) in the contract configuration, the first strut can be on the second side (e.g., bottom) and the second strut can be on the first side (e.g., top) in the expanded configuration.

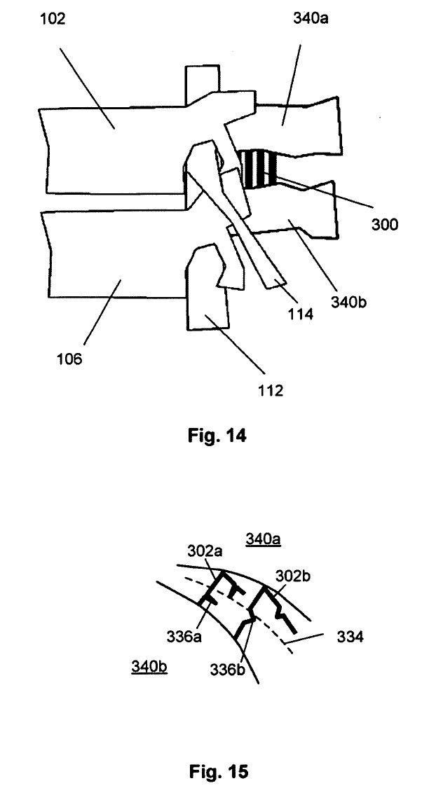

FIG. 14 illustrates that the first vertebra 102 can have a first spinous process 340a and the second vertebra 106 can have a second spinous process 340b. The expandable support device 300 can be deployed between spinous processes 340 on adjacent vertebra. The expandable support device 300 can be deployed between any equivalent peripheral anatomic feature of a vertebra on adjacent vertebrae. For example, the expandable support device can be deployed between adjacent vertebraes' facets, pedicles, laminae, inferior articular precesses, transverse processes, superior articular processes, accessory rocesses, or combinations thereof. More than one expandable support device can be deployed between a first vertebra 102 and a second vertebra 106, for example between different anatomical features on the vertebrae (e.g., between spinous processes and separately between transverse processes).

FIG. 15 illustrates in a partial view of a expandable support device 300 shown close-up deployed between a first spinous process 340a and a second spinous process 340b that the length adjusters 336 on various struts 302 can be expanded and contracted to different lengths, for example to accommodate the surrounding anatomy. For example, first length adjusters 336a on the first strut 302a can be more compressed than the length adjusters 336b on the second strut 302b. The length from the first spinous process 340a to the second spinous process 340b can physiologically be closer at the first strut 302a than at the second strut 302b.

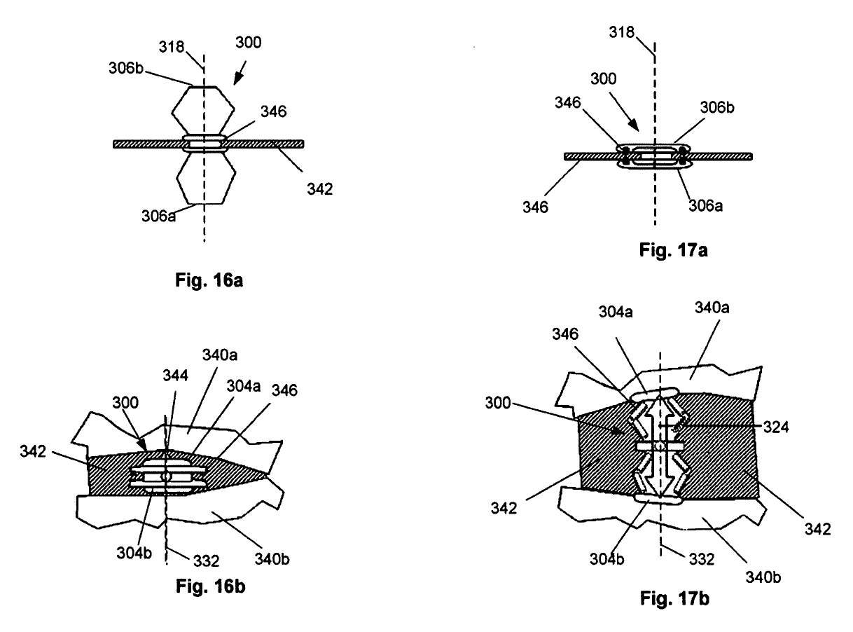

FIGS. 16a and 16b illustrate that the expandable support device 300 can be deployed through a cut or inciscion 344 in soft tissue 342 between the first spinous process 340a and the second spinous process 340b. The cut or inciscion 344 can be performed before the expandable support device is inserted to the target site, and/or by the expandable support device 300, as the expandable support device 300 is inserted to the target site.

The soft tissue 342 can have or be a ligament or tendon. For example, the soft tissue 342 can be the ligamentum flavum, the posterior longitudinal ligament, the anterior longitudinal ligament, or combinations thereof. The deployment tool 338 and/or the expandable support device 300 can have a sharpened distal end, for example configured to cut the soft tissue 342 during deployment.

The expandable support device 330 can be positioned to be on one side of the soft tissue 342 (e.g., the ligament or tendon) or straddle or otherwise be on both sides of the soft tissue 342.

The expandable support device 300 can have tissue attachment elements 346, for example on the struts 302 and or internal or external sides of the plates 304 and/or The tissue attachment devices 346 can be panels, textured surface, hooks, barbs, brads, or combinations thereof.

FIGS. 17a and 17b illustrate that when the expandable support device 300 is expanded, as shown by arrows 324 in FIG. 17b, and longitudinally contracts, the tissue attachment devices 346 can attach to the soft tissue 342 adjacent to the expandable support device 300. As shown in FIG. 17a, the expandable support device 300 can clamp, squeeze, or otherwise attach to the soft tissue 342. The tissue attachment elements 346 can attach to the soft tissue 342. Attachment of the expandable support device 300 to the soft tissue 342 (e.g., via compression of the soft tissue 342 and/or attachment by the tissue attachment elements 346) solely or additionally anchor and/or secure the expandable support device 300.

During expansion and deployment, the top plate 304a can rotate relative to the bottom plate 304b, for example as seen in FIG. 17b. For example, the rotation can occur through flexing or bending in the expandable support device 300.

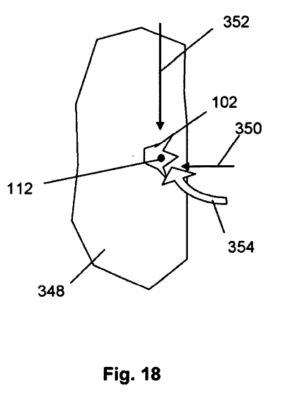

FIG. 18 illustrates paths of inserting the expandable support device 300 through the soft tissue of the back 348 and into the target site, for example adjacent to the first vertebra 102. The expandable support device 300 can be implanted from a posterior approach, as shown by arrow 350, lataral approach, as shown by arrow 352, or a hybrid approach (i.e., mix of posterior and lateral), as shown by arrow 354. The deployed expandable support device 300 can rotate the first vertebra 102 with respect to the second vertebra 106 the equivalent of about the negative vertebral angle 118.

The end plates 306 can indirectly connect more than one strut. The end plates 306 can be in the middle of the length of the expandable support device 300 (i.e., not being “end” plates in that variation) to connect various struts 302 in a transverse plane relative to the longitudinal axis 318.

The expandable support device 300 can have a smaller unexpanded profile than expanded profile. The expandable support device 300 can have a round, square, or rectangular transverse cross section before and/or after expansion.

The expandable support device 300 can have a textured surface, for example, to increase purchase of the bone (e.g., spinous process). The expandable support device 300 can have one or more teeth, serrated surfaces, holes, sharp ridges, or combinations thereof.

The expandable support device 300 can have a tapered shape, for example to increase wedging force applied to the surrounding bone and/or other tissue and/or for better stability to resist migration.

The expandable support device 300 can be porous, for example before or after expansion.

The expandable support device 300 can be mechanically expanded (e.g., deformable), self expanding (e.g., resilient), or both.

The expandable support device 300 can be removed and repositioned from the target site.

The expandable support device 300 can be rigid or have controlled spring force. The device can have support arches. The expandable support device is stabilzed by the soft tissue and creates an interference fit.

The expandable support device 300 does not comprimise the natural soft tissue within the spinal column, this will help create final stability (ligaments are not cut or removed.)

The expandable support device 300 can be curved along a compression and/or longitudinal axis 318.

The expandable support device 300 can have anchors (e.g., sharp points) in the vertebral seats (e.g., bone contact area), for example to securely engage the bone.

The expandable support device 300 can be positioned (e.g., centered over and under the vspinous processes) and/or stabilized by the ligament tissue and bone, during or after deployment of the expandable support device 300.

The expandable support device 300 can be filled/covered with cement, bone, polymer, drug, collagen, or any other agent or material disclosed herein.

The expandable support device 300 can be pre-sized before implantation. The device can be expanded and/or the opposed spinous processes can be distracted with a separate mechanical jack (e.g., distractor or a balloon, such as strong shaped directional balloon). For example, the opposed spinous processes can be distracted before the expandable support device 300 is implanted in a non-expanded, partially expanded, or fully expanded configuration.

The expandable support device 300 can be locked open, for example to increase radial or height resistance. Once expanded, the expandable support device can be fitted with one or more pins, screws, suture, wire, wedges, filler, or combinations thereof, to increase radial resistance.

The expandable support device 300 can be designed to bend, rotate or otherwise flex (e.g., made of Niti, Ti, polymers), for example, to allow extra motion between the adjacent spinous processes.

Additional embodiments of the expandable support device 300 and methods for use of the expandable support device 300, as well as devices for deploying the expandable support device 300 can include those disclosed for the expandable support device in the following applications which are all incorporated herein in their entireties: PCT Application No. PCT/US2005/034115, filed 21 Sep. 2005; U.S. Provisional Patent Application No. 60/675,543, filed 27 Apr. 2005; PCT Application No. PCT/US2005/034742, filed 26 Sep. 2005; PCT Application No. PCT/US2005/034728, filed 26 Sep. 2005; PCT Application No. PCT/US2005/037126, filed 12 Oct. 2005; U.S. Provisional Patent Application No. 60/723,309, filed 4 Oct. 2005; U.S. Provisional Patent Application No. 60/675,512, filed 27 Apr. 2005; U.S. Provisional Patent Application No. 60/699,577, filed 14 Jul. 2005; and U.S. Provisional Patent Application No. 60/699,576, filed 14 Jul. 2005. The aforementioned spinal lift device can be deployed into the target site, for example, after the tissue in the target site has been removed and/or the target site surfaces have been prepared by the expandable support device 300.

Any or all elements of the expandable support device 300 and/or other devices or apparatuses described herein can be made from, for example, a single or multiple stainless steel alloys, nickel titanium alloys (e.g., Nitinol), cobalt-chrome alloys (e.g., ELGILOY® from Elgin Specialty Metals, Elgin, Ill.; CONICHROME® from Carpenter Metals Corp., Wyomissing, Pa.), nickel-cobalt alloys (e.g., MP35N® from Magellan Industrial Trading Company, Inc., Westport, Conn.), molybdenum alloys (e.g., molybdenum TZM alloy, for example as disclosed in International Pub. No. WO 03/082363 A2, published 9 Oct. 2003, which is herein incorporated by reference in its entirety), tungsten-rhenium alloys, for example, as disclosed in International Pub. No. WO 03/082363, polymers such as polyethylene teraphathalate (PET), polyester (e.g., DACRON® from E.I. Du Pont de Nemours and Company, Wilmington, Del.), poly ester amide (PEA), polypropylene, aromatic polyesters, such as liquid crystal polymers (e.g., Vectran, from Kuraray Co., Ltd., Tokyo, Japan), ultra high molecular weight polyethylene (i.e., extended chain, high-modulus or high-performance polyethylene) fiber and/or yarn (e.g., SPECTRA® Fiber and SPECTRA® Guard, from Honeywell International, Inc., Morris Township, N.J., or DYNEEMA® from Royal DSM N.V., Heerlen, the Netherlands), polytetrafluoroethylene (PTFE), expanded PTFE (ePTFE), polyether ketone (PEK), polyether ether ketone (PEEK), poly ether ketone ketone (PEKK) (also poly aryl ether ketone ketone), nylon, polyether-block co-polyamide polymers (e.g., PEBAX® from ATOFINA, Paris, France), aliphatic polyether polyurethanes (e.g., TECOFLEX® from Thermedics Polymer Products, Wilmington, Mass.), polyvinyl chloride (PVC), polyurethane, thermoplastic, fluorinated ethylene propylene (FEP), absorbable or resorbable polymers such as polyglycolic acid (PGA), poly-L-glycolic acid (PLGA), polylactic acid (PLA), poly-L-lactic acid (PLLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, a biomaterial (e.g., cadaver tissue, collagen, allograft, autograft, xenograft, bone cement, morselized bone, osteogenic powder, beads of bone) any of the other materials listed herein or combinations thereof. Examples of radiopaque materials are barium sulfate, zinc oxide, titanium, stainless steel, nickel-titanium alloys, tantalum and gold.

Any or all elements of the expandable support device 300 and/or other devices or apparatuses described herein, can be, have, and/or be completely or partially coated with agents and/or a matrix a matrix for cell ingrowth or used with a fabric, for example a covering (not shown) that acts as a matrix for cell ingrowth. The matrix and/or fabric can be, for example, polyester (e.g., DACRON® from E.I. Du Pont de Nemours and Company, Wilmington, Del.), poly ester amide (PEA), polypropylene, PTFE, ePTFE, nylon, extruded collagen, silicone, any other material disclosed herein, or combinations thereof.

The expandable support device 300 and/or elements of the expandable support device 300 and/or other devices or apparatuses described herein and/or the fabric can be filled, coated, layered and/or otherwise made with and/or from cements, fillers, glues, and/or an agent delivery matrix known to one having ordinary skill in the art and/or a therapeutic and/or diagnostic agent. Any of these cements and/or fillers and/or glues can be osteogenic and osteoinductive growth factors.

Examples of such cements and/or fillers includes bone chips, demineralized bone matrix (DBM), calcium sulfate, coralline hydroxyapatite, biocoral, tricalcium phosphate, calcium phosphate, polymethyl methacrylate (PMMA), biodegradable ceramics, bioactive glasses, hyaluronic acid, lactoferrin, bone morphogenic proteins (BMPs) such as recombinant human bone morphogenetic proteins (rhBMPs), other materials described herein, or combinations thereof.

The agents within these matrices can include any agent disclosed herein or combinations thereof, including radioactive materials; radiopaque materials; cytogenic agents; cytotoxic agents; cytostatic agents; thrombogenic agents, for example polyurethane, cellulose acetate polymer mixed with bismuth trioxide, and ethylene vinyl alcohol; lubricious, hydrophilic materials; phosphor cholene; anti-inflammatory agents, for example non-steroidal anti-inflammatories (NSAIDs) such as cyclooxygenase-1 (COX-1) inhibitors (e.g., acetylsalicylic acid, for example ASPIRIN® from Bayer AG, Leverkusen, Germany; ibuprofen, for example ADVIL® from Wyeth, Collegeville, Pa.; indomethacin; mefenamic acid), COX-2 inhibitors (e.g., VIOXX® from Merck & Co., Inc., Whitehouse Station, N.J.; CELEBREX® from Pharmacia Corp., Peapack, N.J.; COX-1 inhibitors); immunosuppressive agents, for example Sirolimus (RAPAMUNE® from Wyeth, Collegeville, Pa.), or matrix metalloproteinase (MMP) inhibitors (e.g., tetracycline and tetracycline derivatives) that act early within the pathways of an inflammatory response. Examples of other agents are provided in Walton et al, Inhibition of Prostoglandin E₂ Synthesis in Abdominal Aortic Aneurysms, Circulation, Jul. 6, 1999, 48-54; Tambiah et al, Provocation of Experimental Aortic Inflammation Mediators and Chlamydia Pneumoniae, Brit. J. Surgery 88 (7), 935-940; Franklin et al, Uptake of Tetracycline by Aortic Aneurysm Wall and Its Effect on Inflammation and Proteolysis, Brit. J Surgery 86 (6), 771-775; Xu et al, Sp1 Increases Expression of Cyclooxygenase-2 in Hypoxic Vascular Endothelium, J. Biological Chemistry 275 (32) 24583-24589; and Pyo et al, Targeted Gene Disruption of Matrix Metalloproteinase-9 (Gelatinase B) Suppresses Development of Experimental Abdominal Aortic Aneurysms, J. Clinical Investigation 105 (11), 1641-1649 which are all incorporated by reference in their entireties.

It is apparent to one skilled in the art that various changes and modifications can be made to this disclosure, and equivalents employed, without departing from the spirit and scope of the invention. Elements shown with any embodiment are exemplary for the specific embodiment and can be in used on or in combination with other embodiments within this disclosure.

I claim:

1. A method for treating spinal stenosis, comprising:
positioning an expandable support device between a first vertebra and a second vertebra, wherein the first vertebra is adjacent to the second vertebra; and
compressing the expandable support device.

2. The method of claim 1, wherein compressing comprises applying a compressive force in a first direction, and wherein compressing further comprises expanding the expandable support device in a second direction.

3. The method of claim 2, wherein the second direction is substantially perpendicular to the first direction.

4. The method of claim 1, wherein compressing comprises applying a compressive force along an axis that is substantially perpendicular to a line from an anatomical landmark on the first vertebra to the anatomical landmark on the second vertebra.

5. The method of claim 1, wherein compressing comprises expanding the height of the expandable support device.

6. The method of claim 1, wherein the height is measured along an axis that is substantially parallel with a line from an anatomical landmark on the first vertebra to the anatomical landmark on the second vertebra.

7. The method of claim 1, wherein compressing comprises applying a compressive force along an axis that is substantially perpendicular to a line from an anatomical landmark on the first vertebra to the anatomical landmark on the second vertebra.

8. The method of claim 1, further comprising sensing the compressed expandable support device, then further compressing the compressed expandable support device.

9. The method of claim 8, wherein sensing comprises visualizing.

10. The method of claim 1, further comprising sensing the compressed expandable support device, then further expanding the expandable support device.

11. The method of claim 10, wherein sensing comprises visualizing.

12. An expandable support device for treating spinal stenosis by applying substantially oppositely directed forces on a first bone and a second bone, comprising:
an expandable frame comprising: a first elongated element, a second elongated element, and a first connector;
wherein the first elongated element has a first elongated element first end and a first elongated element second end, and wherein the second elongated element has a second elongated element first end and a second elongated element second end, and wherein the first connector connects the first elongated element to the second elongated element, and wherein the expandable frame is configured to expand in a first direction when the expandable frame is compressed in a second direction.

13. The device of claim 12, wherein the first elongated element and the second elongated element interdigitate.

14. The device of claim 12, further comprising a second connector connecting the first elongated element to the second elongated element.

15. The device of claim 12, wherein the first connector is connected to the first elongated element at the first elongated element first end.

16. The device of claim 15, wherein the second connector is connected to the first elongated element at the first elongated element second end.

17. The device of claim 12, wherein the connection between the first elongated element and the first connector comprises the first connector being integral with the first elongated element.

18. The device of claim 12, wherein the first connector is configured to attach to a compression tool.

19. The device of claim 18, wherein the second connector is configured to attach to the compression tool.

20. The device of claim 12, wherein the expandable frame is configured to bend about an axis substantially parallel with the first direction.

21. The device of claim 12, wherein the expandable frame is configured to bend about an axis substantially perpendicular to the first direction and the second direction.

22. The device of claim 12, wherein the first elongated element comprises a seat configured to attach to the first bone, and wherein the seat is configured in a different shape than the adjacent portion of the first elongated element.
